(19) United States
(12) Patent Application Publication
(10) Pub. No.: US 2022/0370714 A1
(43) Pub. Date: Nov. 24, 2022

(54) TISSUE SATURATION RESPONSIVE RAPID AUTOMATICALLY VARIABLE FLOW RATE INFUSION SYSTEM

(71) Applicant: INNOVATIVE HEALTH SCIENCES, LLC, Chester, NY (US)

(72) Inventor: Andrew SEALFON, Chester, NY (US)

(21) Appl. No.: 17/761,410

(22) PCT Filed: Sep. 18, 2020
(86) PCT No.: PCT/US2020/051628

§ 371 Date: Mar. 17, 2022

Related U.S. Application Data
(60) Provisional application No. 62/902,750, filed on Sep. 19, 2019.

(51) Int. Cl. A61M 5/168; A61M 39/10; A61M 39/12; A61M 5/142; A61M 5/158; A61M 5/32

(52) U.S. Cl. CPC A61M 5/16877; A61M 39/105; A61M 39/12; A61M 5/14248; A61M 5/158; A61M 5/3293; A61M 5/3298

(57) ABSTRACT

Infusion systems and methods for administering an infusion fluid into a patients anatomic space at a variable flow rate without flow control include an administration set having a flexible tube fluidically connected to a needle connector, the needle connector including a receiving end fluidically connected to flexible tube, and an administering end opposite the receiving end and fluidically connected with an infusion needle, and the infusion needle having an inside diameter of about 0.0104 inches to about 0.0135 inches and fluidically connected to the administering end of the flexible tubing to deliver the infusion fluid to the patients anatomic space at variable flow rates dependent upon the saturation of the infusion fluid at the patients injection site.

### TECHNICAL FIELD

The invention relates generally to infusion systems, and more particularly, to responsive automatically variable flow rate infusion systems and methods to deliver an infusion fluid at a variable flow rate without flow control.

### BACKGROUND

An infusion pump is a medical device that delivers fluids, including nutrients and medications, into a patient in controlled amounts. The nutrients and medications can include insulin, hormones, antibiotics, chemotherapy drugs, pain relievers, and other fluids. Infusion pumps can be used to deliver fluids intravenously, as well as subcutaneously (beneath the skin), arterially, and epidurally (within the surface of the central nervous system). Infusion pumps can reliably administer fluids in ways that would be impractically expensive, unsafe, or unreliable if performed manually by a nursing staff. Infusion pumps offer advantages over manual administration of fluids, including the ability to deliver fluids in very small volumes and the ability to deliver fluids at precisely programmed rates or automated intervals. For example, infusion pumps can administer 1 ml per hour injections (too small a dose for a drip), injections every minute, injections with repeated boluses requested by the patient, e.g., for patient controlled analgesia (up to a maximum number allowed over a time period), or fluids whose volumes vary by the time of day.

Infusion pump systems often use disposable infusion sets to link the pump system to an infusion site of a patient. These sets usually have tubing between the infusion site and the infusion pump. For constant flow pump systems, the tubing is referred to as an extension set with undefined flow properties. For mechanical pumps, the tubing needs to be modified to obtain desired flow rates with different drug and needle set combinations, as well as accommodate patient movement and position.

Infusion pumps are frequently used to deliver and administer critical fluids, including high-risk medications, so pump failures can have significant consequences for patient safety. Many infusion pumps are fitted with safety features, including alarms and other operator alerts that activate in the event of a problematic incident. Some pumps alert users when air is present or an over-pressure condition is detected in the tubing or catheter that delivers the infusion fluid to the patient. Smart infusion pumps alert the user when there is a risk of an adverse drug interaction, or when the user programs the operating parameters outside of specified safety limits.

While some adverse treatment events may be the result of user error, many of the reported adverse events are related to deficiencies in infusion system design and engineering. These deficiencies can create problems themselves or contribute to user error. The most common types of reported subcutaneous adverse reactions have been associated with needle placement, volume of drug per site, flow rate per site, user errors, and the delivery of fluids with excessive pressures.

Other uses of infusion pump systems can cause discomfort or safety concerns for the patient due to excessive pressure in the system, such as when an obstruction in the infusion fluid delivery is encountered or when the infused liquid saturates the anatomic space. A deficiency of current infusion pump systems is their inability to adjust the flow rate and pressure of the fluid to compensate for changes in pressure and resistance throughout the system.

One of the problems with conventional constant flow rate infusion pump systems is that they deliver the infused liquid at high pressures regardless of patient anatomy or conditions, which can cause injury and/or complications.

More specifically, infusion systems have been developed around the idea that the infusion pump (usually electric infusion pumps) will create volumetric flow rates to deliver medications. If there is any resistance within the fluid path, or inside the patient, the infusion pump will overpower the resistance to force the programmed flow rate until an arbitrary maximum pressure is detected, at which time the pump will shut down and sound an (occlusion) alarm. This is considered an overpressure alarm that activates after the patient has been exposed to the pressure build at which point the patient is at risk to harm.

The infusion industry and Food and Drug Administration (FDA) focus on the flow rate of drug infused and not the pressure at which the drug is infused. All drugs are approved for a particular volumetric dose per unit time. Usually this is designated in milliliters per hour and is referenced back to milligrams per hour or the total mass of drug being delivered to the patient. Infusion systems have been designed with little attention to the resistances of the infusion peripherals and there is scant attention given to the implications of pressure and how it relates to flow rate. This is a common and potentially dangerous industry error.

Prior mechanical infusion systems have attempted to deliver high flow at low pressures but operate typically at about 13.5 psi and require series calibrated flow rate tubing or restrictions to limit flow rates at some pre-set maximum flow rate. See US Patent Publication Number U52019/0201620, for example. These systems operate at a mid-range of pressures (e.g., 10-14 psi) but have made only marginal improvements compared to electric pumps. Previous systems and manufacturers appear resigned to increase the diameter of the needle set tubing to increase the maximum flow rates realized.

The shortcomings of previous systems and methods are illustrated in an example use case during the worldwide COVID-19 crisis. To reduce exposure to the virus, clinicians decided to place the infusion pumps outside of the room of the infected patient, which mitigated the need for a hazmat suit to monitor the patient's infusion within the room. The medical recommendation suggests using tubing with the smallest diameter available, so as to minimize the residual volume of the drug in the system. In most cases, these tubing were 10-15 feet long, some even longer. While clinicians observed that they incurred many more “occlusion” alarms with this method (and they increased the alarm pressure if possible), they did not associate the increased complications of these sick patients with the use of this procedure. To explain, the small diameter of the very long tubing of the extension set actually caused the pump pressure to increase dramatically in efforts to have the drug maintain the flow rate desired and still traverse a longer distance through restrictive tubing to reach the patient.

As long as no blockages of any kind were apparent in the patient, this method would work well. However, if the patient had any issue in their vein, for example: a collapsed vein, a clot, or a thrombosis then the full pump operating pressure (˜30 psi-50 psi) would impinge upon the blockage and essentially blow out the vein. The infusion pump itself would continue to operate normally, giving no sign or indication of alarm—unaware to the dangerous effect created by this method of circumventing exposure to the COVID-19 virus.

Conventional constant flow rate infusion (electric) pump systems failed to deliver the infused liquid at safe pressures under these conditions, leading to potential patient injury and complications.

The above information disclosed in this Background section is only for understanding of the background of the inventive concepts, and, therefore, it may contain information that does not constitute prior art.

### SUMMARY

Variable flow rate infusion systems and methods constructed according to the principles and exemplary implementations of the invention can deliver infusion fluids within a safe pressure range without flow control and with the flow rate being as fast as physically possible or tolerable by the patient (i.e., rapid). In exemplary implementations of the invention, the flow rate changes (variable) as the tissue becomes saturated with drug (tissue saturation responsive) while using safe low pressures.

In infusion systems and methods constructed according to the principles and some exemplary implementations of the invention, an administration set is manufactured and constructed for a very high flow rate without flow control (e.g., average flow rate of about 40 ml/hr) for a very viscous infusion fluid, such as Hizentra®, for example. The administration set uses a much lower pressure (e.g., about 4 psi to about 9 psi) to deliver the infusion fluid. Past systems operated at pressures too high for a direct connection to the patient and often used flow controls and restrictive tubing to reduce the pressure at the infusion site.

By allowing the flow rate to change instead of the pump pressure, exemplary implementations of the invention can eliminate the harm associated with pressure build-up at injection sites when the tissues are blocked/saturated. In conventional electric pump systems, a pump flow rate (which is typically not representative of the true flow rate delivered to the patient) is set and is maintained. To maintain this flow rate, the pressure is changed to respond to any fluid blockages, site saturation, tubing set blockages, and other conditions that may affect the flow. This (increased) pressure change can create harmful pressure build ups at the injection site causing harm to the patient.

In infusion systems and methods constructed according to the principles and exemplary implementations of the invention do not use a fixed flow rate but allow the flow rate to vary and flow as fast as possible, such that pressure can be limited and not cause harm to the patient. In some exemplary implementations, needle sets are connected directly to the pressure source without any flow control or precision tubing to limit flow. The limit comes from the patient's own body, and the infusion system is allowed to “run free” depending only upon the patient's body being able to accept the drug at the locations of the needles. As the areas under the skin in the subcutaneous space are filled (depots), the back pressure slows the infusion down, but the delivery will continue as the pump pressure gently presses the drug against the subcutaneous tissues to perfuse safely and painlessly, albeit slower. The higher the pressures at the site, the lower the flow rate delivered. This also depends upon the volume of drug used. As the volume increases, more drug fills the depots, and as the rate slows, the delivery time increases.

The industry expects every infusion system to operate at a constant flow rate. The procedure for subcutaneous immunoglobulin administrations, should be the opposite of what is considered acceptable practice for intravenous (IV) administrations. Generally, for IV administrations, the flow rate is started slowly (so as to assure there are no anaphylactic reactions) and then ramped up as indicated in the information package insert for the infused drug. Conversely, for subcutaneous administration, the depots (areas within the patient's subcutaneous tissues) are empty at the beginning of infusion, and then as they fill with a drug, increase in pressure and press against the tissues (extracellular matrix) as the drug is perfused throughout the depot area. As such, the ideal method to infuse large volumes of subcutaneous medications is to begin with as fast a flow rate as possible, and then decrease the flow rate as the depots become saturated. Conventional medical practitioners believe that the flow rate should be constant, which for subcutaneous administration means that the flow rate starts out too slow and then ends up being too fast for the patient.

At low pressures (e.g., below 9 psi), infusion systems and methods constructed according to the principles and some exemplary implementations of the invention can deliver a very viscous fluid (13.7 cP) very quickly, through a very small needle (e.g., 26 gauge) and without causing pain and discomfort. There is no system currently available that can perform this function. Infusion systems and methods constructed according to the principles and some exemplary implementations of the invention can infuse viscous drugs rapidly through a small needle as needle size directly affects patient's perception of pain. In one exemplary implementation, the administration needle sets include inlet tubing, a connection to the needle, and the needle itself. In a direct comparison with commercially available infusion systems, inlet tubing constructed according to the principles and some exemplary implementations of the invention has a larger diameter and the tubing-to-needle connector bridges the size differences between the inlet tubing and the needle to insure a smooth transition (i.e., laminar flow) to the needle set as opposed to the abrupt transition in known commercial systems. The needle itself has a larger internal diameter than that used in known systems.

Infusion systems and methods constructed according to the principles and exemplary implementations of the invention recognize and highlight several unconventional concepts, including those referenced above, which provide technological solutions to problems with existing systems. Contrary to accepted medical practice, exemplary implementations of the invention recognize and address the findings that It is pressure that causes most of the adverse site reactions in the subcutaneous delivery of medications. Infusion systems and methods constructed according to the principles and exemplary implementations of the invention recognize and overcome problems with starting the infusion of immunoglobulins at a slow flow rate and then ramping up to the maximum (new 60 ml/hr/site) flow rates. This technique is extremely inefficient and generally harmful. Further, infusion systems and methods constructed according to the principles and exemplary implementations of the invention incorporate knowledge and practical effects that demonstrate that an effective delivery of medications starts the infusion at the maximum flow rate possible and tapers down the flow rate as the depots fill with fluid.

Contrary to existing infusion systems and methods, infusion systems and methods constructed according to the principles and exemplary implementations of the invention incorporate a recognition that great care and attention is needed to ensure that the infusion circuit path has an absolute minimum resistance to flow.

Infusion systems and methods constructed according to the principles and exemplary implementations of the invention can optimize the fastest delivery rate possible at the lowest pressure possible. The Hagen-Poiseuille fluid flow rate equation posits that the flow rate is directly proportional to pressure and is proportional to the (internal) diameter of the flow path (tube) to the fourth power. Thus, even small increases in the diameter of the administration set result in significant increases in flow rates. This concept, in direct conflict with the principles employed in conventional infusion systems, is incorporated in infusion systems and methods constructed according to the principles and exemplary implementations of the invention to create a fast flow rate at the beginning of the infusion that dramatically tapers off to lower flow rates at the end of the infusion. The overall increase in performance is nearly twice that of known existing systems.

In infusion systems and methods constructed according to the principles and exemplary implementation of the invention, when the depots in the process of saturation and are eventually filled, the operating pressure of the infusion pump system is very low (about 4-9 psi), such that a very low, safe, and painless pressure will gently continue to perfuse the drug into the tissues at a greatly reduced flow rate. This ensures that the patient will eventually obtain the entire dose, but without pain, inflammation, or swelling.

Contrary to generally accepted infusion procedures, the time of the infusion is not controlled by the flow rate of the pump. In a conventional electric pump system, if less time is desired (a patient's personal preference and a quality of life issue), then the clinician will increase the flow rate. However, while the increased flow rate may temporarily appease the patient, there is a high probability that the patient will experience adverse reactions (inflamed skin, pain, etc.) from this method due to the increase in pressure, which results from the drug pushing its way into various places beneath the skin, and possibly inflame mast cells. In infusion systems and methods constructed according to the principles and some exemplary implementation of the invention, if the patient wishes to reduce their infusion time, they will simply add more infusion sites to the administration of the infusion fluid. By adding more sites, there is an increase in perfusion and absorption area. Needle phobic patients who wish to use fewer needle punctures would expect longer times of infusion. Patients who do not mind additional needle sticks are able to significantly reduce their time of infusion.

This concept of adding additional sites solely to decrease administration time is unconventional and not in use or accepted by pharmaceutical companies at this time. According to the principles and some exemplary implementation of the invention, this concept can be implemented using a wearable micro pump connected via a short length of tubing (3 inches) to a nearby needle set that creates the very fast flow rates desired 460 ml/hr/site and greater). Wearable pumps are an attractive option for patients due to their portability and convenience, but they are expensive and limited by needle size, and thus they fall short on performance.

Infusion systems and methods constructed according to the principles and some exemplary implementation of the invention incorporate a tiny wearable pump that is very low in cost and very high in performance. The pumps deliver the fastest flow rates and meet cost targets. The pumps improve quality of life issues and contribute to ease of use and improved infusion experience. These pumps are an attractive alternative to subcutaneous push methods and would automatically deliver the exact fluid volume comfortably and without adverse side reactions that occur often with subcutaneous push methods.

Additional features of the inventive concepts will be set forth in the description which follows, and in part will be apparent from the description, or may be learned by practice of the inventive concepts.

According to one aspect of the invention, an infusion system for administering an infusion fluid into a patient's anatomic space at a variable flow rate without flow control includes an administration set having a flexible tube fluidically connected to a needle connector, the needle connector including a receiving end fluidically connected to flexible tube, and an administering end opposite the receiving end and fluidically connected with an infusion needle, and the infusion needle having an inside diameter of about 0.0104 inches to about 0.0135 inches and being fluidically connected to the administering end of the flexible tubing to deliver the infusion fluid to the patient's anatomic space at variable flow rates dependent upon the saturation of the infusion fluid at the patient's injection site.

The infusion fluid may be at least one of the group of an immunoglobulin, a chemotherapeutic fluid, a monoclonal antibody, an antibiotic, an analgesia fluid, and a hydration fluid.

The flexible tube may have an inside diameter of about 0.039 inches to about 0.045 inches.

The administration set may further include a flexible assembly that conforms to the patient's body to position the needle and reduce the risk of vessel damage.

The infusion system may further include an infusion pump configured to deliver infusion fluid at a variable flow rate and at a pressure of about 4 to about 9 psi.

The infusion pump may be a compact micro syringe infuser, and the administration set may be connected directly to the compact micro syringe infuser.

The compact micro syringe infuser may include a fluid reservoir with a volume of about 5-10 ml, and the administration set may be pre-calibrated and include a length of the tubing measuring about 3 inches to about 7 inches from the micro syringe infuser to the infusion needle.

The infusion pump may be configured to deliver infusion fluid at a variable flow rate and at a pressure of about 4 to about 9 psi.

The needle connector may transition a size difference from the flexible tubing to the needle to continue laminar flow.

The infusion system may be configured to attach to the patient via an adhesive patch.

The infusion system may include a garment configured to secure and house the infusion system and be worn by the patient.

According to another aspect of the invention, a method for administering an infusion fluid into a patient's anatomic space at a variable flow rate without flow control includes the steps of receiving the infusion fluid from an infusion fluid reservoir at a low pressure into flexible tube; delivering the infusion fluid from the flexible tube to a receiving end of a needle connector, the needle connector being configured to maintain laminar flow of the infusion fluid; passing the infusion fluid from the receiving end of the needle connector to an administering end of the needle connector; delivering the infusion fluid from the administering end of the needle connector to an infusion needle; and delivering the infusion fluid to the patient's anatomic space at automatically variable flow rates dependent upon the saturation of the infusion fluid at the patient's anatomic space.

The infusion fluid may be at least one of an immunoglobulin, a chemotherapeutic fluid, a monoclonal antibody, an antibiotic, an analgesia fluid, and a hydration fluid.

The infusion method may further include delivering the infusion fluid to a plurality of infusion needles.

The infusion method may further include using a flexible assembly that conforms to the patient's body to position the needle and reduce the risk of vessel damage.

The infusion method may further include receiving the infusion fluid from an infusion pump with the flexible tube, where the infusion pump is configured to deliver the infusion fluid at a variable flow rate and at a pressure of about 4 to about 9 psi.

The infusion pump may include a compact micro syringe infuser configured to deliver the infusion fluid at a pressure of about 4 to about 9 psi; and the flexible tube may be connected directly to the compact micro syringe infuser.

The compact micro syringe infuser may include a fluid reservoir with a volume of about 5-10 ml; and the combination of the flexible tubing and the infusion needle may be pre-calibrated and include a length of the tubing measuring about 3 inches to about 7 inches from the micro syringe infuser to the infusion needle.

The flexible tube may have an inside diameter of about 0.039 inches to about 0.045 inches and the infusion needle may have an inside diameter of about 0.0104 inches to about 0.0135 inches.

The system may be configured to attach to the patient via an adhesive patch.

The infusion system may include a garment configured to secure and house the infusion system and worn by the patient.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate exemplary embodiments of the invention, and together with the description serve to explain the inventive concepts.

FIG. 1 illustrates an exemplary embodiment of a variable flow rate infusion system having a three-legged needle set constructed according to the principles of the invention for delivering an infusion fluid to patients as part of their infusion therapy.

FIG. 2 illustrates another exemplary embodiment of a variable flow rate infusion system having a one-legged needle set constructed according to the principles of the invention for delivering an infusion fluid to patients as part of their infusion therapy.

FIGS. 3A-3D illustrate an exemplary embodiment of a needle connector constructed according to the principles of the invention for delivering an infusion fluid to patients as part of their infusion therapy.

FIG. 4 illustrates an exemplary embodiment of an infusion pump used in a variable flow rate infusion system constructed according to the principles of the invention, where the infusion pump is a micro syringe infuser.

FIG. 5 illustrates an exemplary embodiment of an infusion pump used in a variable flow rate infusion system constructed according to the principles of the invention, where the infusion pump is worn on a garment.

FIG. 6 illustrates an exemplary embodiment of an infusion pump used in a variable flow rate infusion system constructed according to the principles of the invention, where the infusion pump is attached to the patient with an adhesive patch.

### DETAILED DESCRIPTION

In the following description, for the purposes of explanation, numerous specific details are set forth in order to provide a thorough understanding of various exemplary embodiments or implementations of the invention. As used herein “embodiments” and “implementations” are interchangeable words that are non-limiting examples of devices or methods employing one or more of the inventive concepts disclosed herein. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments. Further, various exemplary embodiments may be different, but do not have to be exclusive. For example, specific shapes, configurations, and characteristics of an exemplary embodiment may be used or implemented in another exemplary embodiment without departing from the inventive concepts.

Unless otherwise specified, the illustrated exemplary embodiments are to be understood as providing exemplary features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an exemplary embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z—axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various exemplary embodiments are described herein with reference to sectional and/or exploded illustrations that are schematic illustrations of idealized exemplary embodiments and/or intermediate structures. As such, variations from the shapes of the illustrations as a result, for example, of manufacturing techniques and/or tolerances, are to be expected. Thus, exemplary embodiments disclosed herein should not necessarily be construed as limited to the particular illustrated shapes of regions but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Variable flow rate infusion systems and methods constructed according to the principles and exemplary embodiments of the invention accurately and reproducibly deliver an infusion fluid to a patient at a desired anatomical location by allowing for direct control of the infusion system pressure by the infusion driver (pump). The system pressure is created by the driver alone, and the flow rate of the delivered infusion fluid is determined by the difference in the infusion driver pressure and the site pressure, as detailed further below. In some situations, the pressure, and therefore the infusion system flow, rate may be determined by the patient, who can accept a delivered volume of an infusion liquid at a pressure/speed that does not cause discomfort. For example, a patient can receive the infusion fluid at a flow rate to arrive at a total infusion time while keeping the fluid pressure within a comfortable limit. If the rate is not tolerable, the patient could halt the infusion totally with a slide clamp, wait some time, and restart. This will allow the perfusion to take place without more drug being delivered to the site. Pressures will reduce and it will take longer to infuse the drug, however. Faster flow rates translate to shorter infusion times at higher pressures, while slower flow rates create lower infusion fluid pressures but longer treatment times. With variable flow rate infusion systems and methods according to exemplary embodiments of the invention, a patient or clinician selects this infusion system to minimize the amount of pain or discomfort caused by resistance of fluid-filled tissue space and associated pressures while also increasing the fluid flow rate.

Similarly, a patient or clinician can use needle sets with multiple needles (e.g., from 2-8 needles) in parallel so as to infuse the patient at multiple sites to decrease the overall infusion time. In exemplary embodiments of the invention, no change in flow rate per needle and no change in pressure need to occur.

The maximum pressure of the (pump) driver will always be (equal to or) higher than any other pressure in the system. For example, when an occlusion occurs, the site pressure builds up. When the site pressure equals the driver pressure, the pressure differential is zero, and all (fluid) flow stops. At site pressures lower than pump pressure, some flow will occur.

According to the principles and some exemplary embodiments of the invention, a predetermined maximum pump (driver) pressure may be used to automatically prevent continuous drug (fluid) flow into surrounding tissues if the site system pressure exceeds the maximum or a range of pressures. That is, if the infusion site pressure builds to the predetermined maximum pump pressure, the pump can be shut down or the infusion fluid flow can be manually clamped off at the administration set using side clamps as noted above to stop fluid flow and to resume fluid flow (when the infusion site pressure dissipates) during an infusion. The pressure limits and ranges limit the delivery of an infusion fluid into unintended tissues by eliminating the need for a continuous flow of fluid during the placement of a needle and allow the identification of a fluid-filled space once the resumption of fluid-flow occurs within patient tissues.

### Example System Components

According to one exemplary embodiment of the invention, the variable flow rate infusion system includes an administration needle set that includes inlet tubing, a tubing-to-needle connector, and a needle itself. In one example embodiment, the inlet tubing has a diameter of about 0.040 inches (compared to 0.033 diameter of known systems). The connector smoothly transitions the size difference from the tubing to the needle ensuring laminar flow is maintained. Previous systems included abrupt transitions from tubing to needle and did not maintain laminar flow. Further, the needle in exemplary embodiments of the invention is larger at about 0.0114 inches compared to 0.0098 in one known previous systems (Comparative Embodiment). These increases in inside diameters results in significant increases in flow rates as shown in detail below.

### The Design Principle

Exemplary embodiments of the invention perform their intended function using the Hagen-Poiseuille equation (HPE) to determine the flow rate of a fluid, with some viscosity, given the length and radius of fluid-pathed components (i.e., the needle tubing) within the administration set, and the differential pressure between the pressure source (i.e., the infusion driver) and the patient's infusion anatomic site. To use the HPE, the following assumptions are met: the fluid is incompressible, Newtonian, is not accelerating within the administration set, is in laminar flow through the fluid-pathed components of the administration set that maintain a constant circular cross-sectional area, and has a length that is substantially larger than its diameter.

Given the above, the HPE can be written as:

$$Q = \frac{Δ p π {R}^{4}}{8 L μ}$$

where:

Q is the volumetric flow rate of the infusion fluid

Δp is the differential pressure between the pressure source and the patient's infusion anatomic site

R is the radius of the fluid-pathed component

L is the length of the fluid-pathed component

μ is the dynamic viscosity of the infusion fluid.

An administration set constructed according to some exemplary embodiments of the invention can achieve a low-restriction flow rate through the needle set. Following the HPE, the fluid-pathed components (i.e., the needle tubing) impact the flow rate through their radius (R) and length (L). For simplicity, the flow rate (Q) is proportional to the radius (R) and inversely proportional to the length (L) of the fluid-pathed components. Given the derivation of the HPE (not shown), the radius (R) is to the fourth power (R⁴) demonstrating that the radius (R) substantially affects the flow rate (Q) in comparison to the length (L) of fluid pathed components.

The administration set constructed according to some exemplary embodiments of the invention achieves a low-restriction flow rate through the needle sets, which are comprised of the fluid-pathed components: the needle and the needle tubing, in which both are joined via a needle connector. To achieve a low-restriction flow rate, the needle may have an inside diameter of about 0.0104 inches to about 0.0135 inches and does not exceed a length of about 1.054 inches and the needle tubing has an inside diameter of about 0.039 inches to about 0.045 inches with lengths that may be about 3 inches to about 7 inches and about 18 inches to about 26 inches.

The differential pressure (Δp) of the system is the pressure drop of the system along the fluid-pathed components of the administration set between pressure source (i.e., the infusion driver) and the patient's infusion anatomic site. For simplicity, the flow rate (Q) is proportional to the differential pressure (Δp).

In one simplified example, a patient prepares for an infusion for the subcutaneous delivery of an infusion fluid using a constant pressure source, 5 psi for example, to deliver the infusion fluid. Assuming very low or no pressure, 0 psi for example, at the patient's infusion anatomic site at the beginning of the infusion the flow rate is as fast as possible, following the principles of the HPE. The differential pressure (Δp) is:

(Δp)=Pressure_{Pressure Source}−Pressure_{Anatomic Site}

such that in this example Δp=5 psi−0 psi=5 psi.

At a point later in the infusion, the patient's infusion anatomic site will begin to fill with the infusion fluid. This is called site saturation. As the anatomic site saturates, the pressure at the anatomic site increases—a natural biological function. As the rate of infusion may exceed the rate of infusion fluid perfusion into nearby spaces (i.e., extracellular matrix) at the anatomic site, the pressure will increase. At this point later in the infusion, the pressure at the anatomic site will now be higher than previous, 1 psi for example. Now, the differential pressure (Δp) is Δp=5 psi−1 psi=4 psi.

The initial differential pressure (Δp) was 5 psi at the start of the infusion and 4 psi at some point later on during the infusion. Given, all other HPE values remain constant, only the flow rate (Q) can change in response to the differential pressure (Δp). For simplicity, the differential pressure (Δp) is proportional to the flow rate (Q), and the pressure at the anatomic site is proportional to site saturation. As such, as the pressure at the anatomic site increases, the differential pressure (Δp) decreases and the flow rate (Q) decreases. As the infusion progresses, the patient's infusion anatomic site is increasingly filled and as such, flow rate (Q) decreases. As a result, the flow rate (Q) changes throughout the infusion.

Intrinsically, the specifications of fluid-pathed components (i.e., the needle and needle tubing) do not constitute “low-restriction flow rate administration sets” on their own. The flow rates (Q) of each fluid-pathed component must be combined to determine the total flow rate of the administration set. This may be done using the following total flow equation (TFE) (derivation not shown):

$${Q}_{T otal Flow Rate} = \frac{( {Q}_{Needle} ) ( {Q}_{Needle Tubing} )}{( {Q}_{Needle} + {Q}_{Needle Tubing} )}$$

where:

Q_{Total Flow Rate} is the total flow rate of the administration set

Q_{Needle} is the flow rate of the needle

Q_{Needle Tubing} is the flow rate of the needle tubing.

Here, Q_{Needle} and Q_{Needle Tubing} can be calculated using the HPE as mentioned earlier. In the case of administration sets with multiple legs of needle and needle tubing, the total flow rate of administration set can be calculated by:

Q_{Total Flow Rate Multiple Legs}=(n)(Q_{Total Flow Rate})

where:
Q_{Total Flow Rate Multiple Legs} is the total flow of the administration set containing more than one needle and needle tubing, collectively called a “needle leg”
N is the number of needle legs in the administration set
Q_{Total Flow Rate} is the total flow rate of the administration set.

Administration sets constructed according to some exemplary embodiments of the invention may be compared to previous systems also claiming a low-restriction flow rate administration sets, such as those presented in US Patent Publication Number 2019/0201620 and/or known systems discussed above.

The following example compares an administration set constructed according to an exemplary embodiment of the invention of the invention (“IHS Set”) to the administration set of the Comparative Embodiment, with regard to flow rate performance. In the example, the same infusion fluid, with a dynamic viscosity (μ) of 13.7 cP (the viscosity of Hizentra®) is delivered through each administration set, an experimental differential pressure (Δp) of 13.5 psi is used, and all other infusion parameters are the same for both administration sets. As such, the flow performance (Q) is determined by the radius (R) and length (L) of the fluid-pathed components—the needle and needle tubing. As the radius (R) is raised to the fourth power (R⁴) it substantially affects the flow rate (Q) more than length (L) for both the needle and the needle tubing and as such, the length (L) is negligible in this simplified example, and is an experimental length value of 1 inch for the needle and 24 inches for the needle tubing for both IHS Set and Comparative Embodiment administration sets. The true diameters for the needle and needle tubing of both administration sets is used. In some exemplary embodiments of the invention, the IHS Set has a needle diameter of 0.0110 inches and a needle tubing diameter of 0.040 inches. The Comparative Embodiment has a needle diameter of 0.0098 inches and a needle tubing diameter of 0.033 inches. Now, using the HPE to determine flow rates (Q):

### IHS Set

$${Q}_{IHS Set Needle} = \frac{( 13.5 PSI ) π {( \frac{{0.011}^{″}}{2} )}^{4}}{8 ( {1}^{″} ) ( 13.7 cP )} = ∼ 145 ml / hr$$

$${Q}_{IHS Set Needle Tubing} = \frac{( 13.5 PSI ) π {( \frac{{0.04}^{″}}{2} )}^{4}}{8 ( {24}^{″} ) ( 13.7 cP )} = ∼ 1050 ml / hr$$

### Comparative Embodiment

$${Q}_{Super 26 Needle} = \frac{( 13.5 PSI ) π {( \frac{0 {\text{.0098}}^{″}}{2} )}^{4}}{8 ( {1}^{″} ) ( 13.7 cP )} = ∼ 90 ml / hr$$

$${Q}_{Super 26 Needle Tubing} = \frac{( 13.5 PSI ) π {( \frac{{0.033}^{″}}{2} )}^{4}}{8 ( {24}^{″} ) ( 13.7 cP )} = ∼ 490 ml / hr$$

The flow rates (Q) of each individual fluid-pathed component are calculated for each set using the HPE, shown above. Now, the total flow rate of each administration set is calculated (both sets are assumed to only have one needle and needle tubing each in this example). Now, using the TFE determine total flow rate (Q_{Total Flow Rate}):

### IHS Set

$${Q}_{Total Flow Rate IHS Set} = \frac{( 145 ml / hr ) ( 1050 ml / hr )}{( 145 ml / hr ) + 1050 ml / hr )} = ∼ 130 ml / hr$$

### Comparative Embodiment

$${Q}_{Total Flow Rate Super 26} = \frac{( 90 ml / hr ) ( 490 ml / hr )}{( 90 ml / hr ) + 490 ml / hr )} = ∼ 80 ml / hr$$

The IHS Set flow rate performance is substantially faster than that of the Comparative Embodiment and as such is considered a low-restrictive flow rate tubing set that, in combination with a other infusion system components constructed according to the principles and exemplary embodiments of the invention, enables delivery of fast flow rates to the patient's anatomic site.

### Additional System Components

Exemplary embodiments of the invention can also include a fluid reservoir (e.g., an infusion fluid storage device such as a syringe, a bag, or other reservoir), an infusion fluid (such as an immunoglobulin), and an infusion pump. An infusion fluid flow rate and volume per site are determined based on patient variables (e.g., diagnosis, size, weight, location of infusion, etc.) and infusion therapy variables (e.g., infusion fluid viscosity, volume of infusion fluid, needle size, tubing diameter, tubing length, infusion rates of the fluid, etc.).

According to one exemplary embodiment of the invention, the infusion pump may be a micro-pump or syringe driving device such as an infusion driver constructed according to the principles and the exemplary embodiments disclosed in the Assignee's co-pending US. Patent Application No. ______, entitled, “Systems and Methods for Precision Matched Immunoglobulin Infusion,” (Attorney docket no. 213.0001-US00) filed simultaneously herewith, the disclosure of which is incorporated by reference herein in its entirety. According to one exemplary embodiment of the invention, the infusion pump is a constant pressure/force pump to provide a constant pump pressure. In some exemplary embodiments, the infusion pump is set at a pressure of 5 psi. A target flow rate, which may be the infusion fluid flow rate, is identified and is allowed to vary during the infusion therapy treatment session as long as the fluid pressure is equal to or below the maximum infusion fluid pressure. As discussed above, the flow rate may slow (i.e., vary) as the infusion site pressure builds due to site saturation, but the infusion treatment continues, and some flow will occur, as long as the site pressure remains below the maximum pressure.

The flow rate is allowed to vary based on the pre-set maximum infusion fluid pressure during treatment. As long as the infusion fluid pressure is below the pre-set, low maximum pressure, the fluid flow rate is not as important. In some exemplary embodiments of the invention, the pre-set pump maximum pressure is a pressure between about 4 psi (200 mmHg) and about 9 psi (465 mmHg).

Variable flow rate infusion systems and methods according to the principles and exemplary embodiments of the invention provide safe and effective means for delivering a rapid infusion fluid flow rate using a fluid reservoir, an infusion fluid, an infusion pump, and a needle set to deliver the infusion fluid from the reservoir into the patient (via the needle set). According to some exemplary embodiments, the system includes a pump that delivers a infusion fluid at a fixed pressure and allows the flow rate of the infusion fluid to vary as the infusion site fills or saturates, as long as the patient's anatomic site pressure is less than the infusion pump's fixed pressure. The system generally does not require any patient or clinician interaction as the pump pressure produces a maximum pump pressure (e.g., 5 psi), and, when combined with a very low-restriction needle set, produces a maximum flow rate which is reduced by any increases in the patient's site pressure.

In one exemplary embodiment of the invention, the infusion fluid pressure (resistance measure) can be displayed visually or presented audibly, such as in a read-out or a continuous audible signal, respectively, when the pressure (resistance measure) is within or outside the pre-set range. The patient or clinician can monitor the pressure measurements to determine and/or confirm that the infusion fluid is being delivered correctly. The pump controller can record and track performance parameters for future review and correlation and adjust the pressure at the pump accordingly. In other exemplary embodiments of the invention, upon calculating the force readings, the pressure limit(s) can be pre-defined to ensure that excessive pressures are not reached.

### Example Infusion System Operation

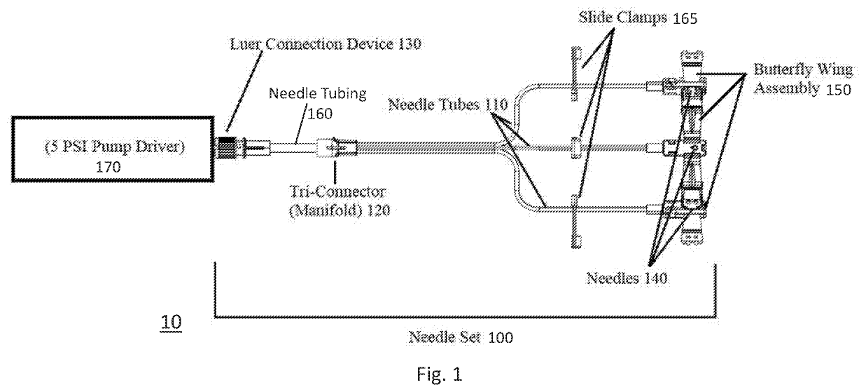

FIG. 1 illustrates a variable flow rate infusion system 10 without flow control having a needle set 100 constructed according to an exemplary embodiment of the invention. Needle set 100 includes a flexible needle positioning assembly that conforms to the patient's body to position the needle and reduce the risk of vessel damage. In some exemplary embodiments of the invention, the flexible assemblies are butterfly wing assemblies 150. Needle set 100 also includes multiple needles 140. In the exemplary embodiment of the invention shown in FIG. 1, the needles 140 are configured in a parallel configuration. In the exemplary embodiment, three needles are shown. Further, the needles 140 have a 90° needle angle to be placed subcutaneously, as deep as required specific to the patient. The needle set 100 can include a connection device 130, such as a Luer lock, for example, to connect the needle tubing 160 to the infusion pump. This exemplary embodiment contemplates that needles generally will be available with sharpened administering lengths of 6 mm, 9 mm, 12 mm and 14 mm, which depend on the thickness of the adipose tissue of the patient to ensure the fluid reaches the subcutaneous space. The needle set 100 includes needle tubing 160 that extends from the Luer lock 130 to a manifold 120 where the needle set tubing 160 divides into individual needle tubes 110. For convenience and brevity, three individual needle tubes 110 are shown in FIG. 1, but additional needle tubes 110 and needles 140 can be used. Similarly, additional butterfly 150 can also be used with each needle 140 placed on its own butterfly 150. The needle set is configured to deliver infusion fluid at a variable flow rate with low pressures.

According to exemplary embodiments of the invention, a maximum infusion fluid pressure of 5 psi is determined to be a safe and effective pressure for a patient. In other exemplary embodiments of the invention, the maximum infusion fluid pressure may be from about 4 psi to about 7 psi, as high as 9 psi. For a variable flow needle set without a series flow restriction, fluid pressures above 9 psi could be too high and dangerous to the patient. The maximum infusion fluid pressure determination is based on patient variables, patient status (e.g. naïve or experienced), the size and weight of the patient, and the location of the infusion site (e.g., subcutaneous abdominal, and other patient variables). The maximum infusion fluid pressure of 5 psi is also based on infusion therapy variables, including infusion fluid viscosity, volume of infusion fluid, needle size, needle set tubing diameters, needle set tubing lengths, infusion rates of the fluid, and other infusion therapy variables. A target flow rate is identified based on the volume of infusion fluid to be delivered and the amount of time the infusion therapy treatment will take. In some exemplary embodiments of the invention, the target flow rate is between about 5 ml/hr and 125 ml/hr. In the example of FIG. 1, the volume of infusion fluid is 60 ml, and the patient has 1 hour to complete the therapy. The target flow rate in this example is determined to be 60 ml/hr, but the actual flow rate is allowed to vary during the infusion therapy treatment session as long as the infusion fluid pressure is equal to or below the predetermined maximum infusion fluid pressure (e.g., 5 psi). An example of a very high flow rate without flow control would be an average flow rate of about 40 ml/hr for a very viscous infusion fluid, such as Hizentra®, for example.

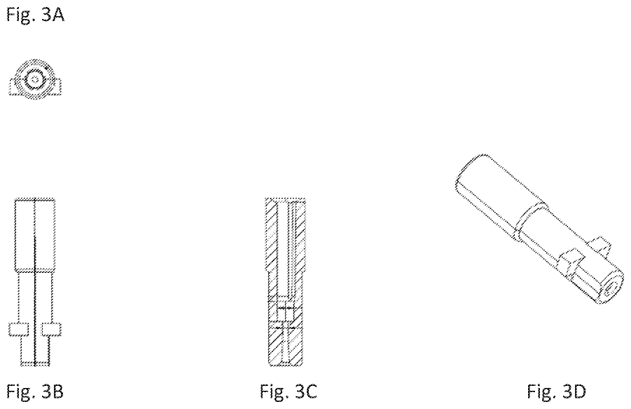

The geometries of the needle set are selected based the maximum flow rate at the lowest pressure. Other variables may include needle material properties (e.g., thin wall stability), needle dimensions, and fluid properties (e.g., viscosity and infusion rate). For example, in one exemplary embodiment of the invention, the needles are about 1 inch long and are bent to expose about 6-14 mm. In some exemplary embodiments, the needle may have an inside diameter of about 0.0104 inches to about 0.0135 inches and does not exceed a length of about 1.054 inches, and the needle tubing has an inside diameter of about 0.039 inches to about 0.045 inches with lengths that may be about 3 inches to about 7 inches and about 18 inches to about 26 inches. In one exemplary embodiment of the invention, a needle is fluidically connected to the needle tubing via a needle connector (see FIGS. 3A-3D, for example). The outside diameter of the needle tubing (about 0.0551 inches to about 0.0787 inches) fits within one end's inside diameter (about 0.060 inches to about 0.080 inches) of the needle connector. The outside diameter of the needle (about 0.0175 inches to about 0.0228 inches) at the receiving end fits within the other end's inside diameter of the needle connector (about 0.0200 inches to about 0.0275 inches). The inside diameter of the needle connector can be sized and manufactured to house 26-24G needles with required diameters needed to fit about 0.0175 inches to about 0.0228 inches outside diameter needles. In one exemplary embodiment a needle connector is sized and manufactured for a needle with an outside diameter of 0.0181 inches and a needle tubing with outside diameter of 0.0677 inches. The inside diameters of both sides of the needle connector taper down so that glue can be filled in the gap to secure the structure and prevent the infusion fluid from leaking. Additionally, the needle seat can sit inside a butterfly assembly, such as that disclosed in the Assignee's co-pending U.S. patent application Ser. No. ______, entitled, “Systems and Methods for Precision Matched Immunoglobulin Infusion,” (Attorney docket no. 213.0001-US00) filed simultaneously herewith, the disclosure of which is incorporated by reference herein in its entirety.

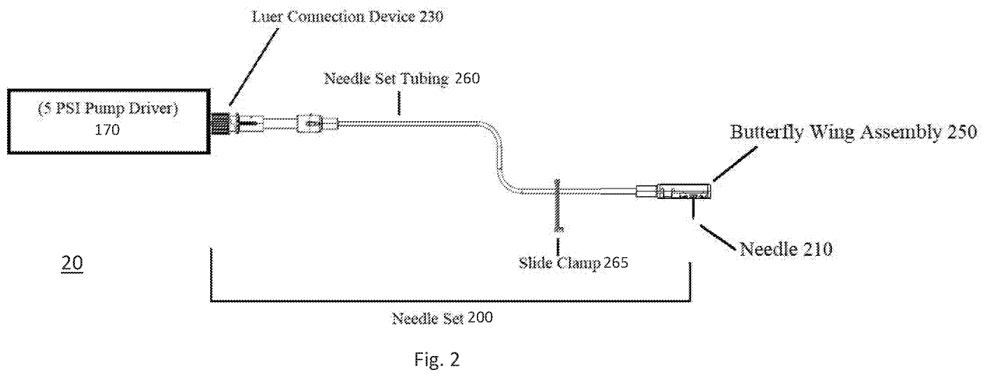

FIG. 2 illustrates another variable flow rate infusion system 20 constructed according to an exemplary embodiment of the invention. Needle set 200 includes a single butterfly wing assembly 250 and a single infusion needle 210. In the exemplary embodiment of the invention shown in FIG. 2, the needle 210 is positioned on a patient's anatomical space to deliver immunoglobulin to a single site. In another exemplary embodiment (not shown separately), the needle 210 includes more additional needles configured in a parallel configuration. The needle set 200 of FIG. 2 can include a connection device 230, such as a Luer lock, for example to connect to the infusion pump. The needle set 200 includes needle set tubing 260 that extends from the Luer lock 230 to the needle 210. Depending upon the size of the needle 210, patient preference, and the anatomical sites to which the infusion fluid is to be delivered, additional butterfly wing assemblies 250 can also be used with additional needles 210. In the exemplary embodiment shown in FIG. 2, the maximum infusion fluid pressure is determined similarly to the manner of FIG. 1. Likewise, a target flow rate is identified in a similar manner, as are the needle set component geometries.

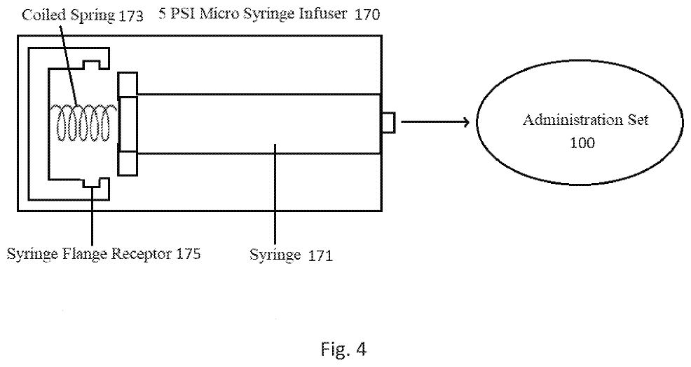

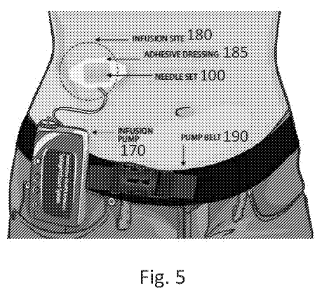

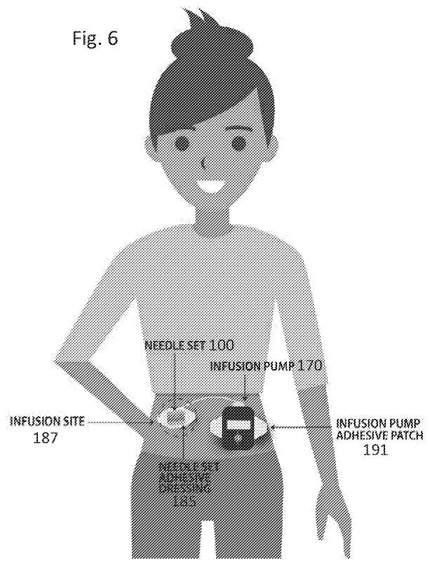

In one exemplary embodiment of the invention, the infusion pump 170 is syringe driver, such as a compact micro syringe infuser, and the needle set 200 is connected directly in a compact and low-cost disposable system worn by the patient. The volume of the micro syringe infuser is 5-10 ml, and the length of the tubing to the needle is from about 3 inches to about 7 inches. In this compact system, the micro syringe infuser is connected directly to the needle set tubing 260 at one end and directly to the needle 210 at the opposite end. As shown in an exemplary embodiment illustrated in FIG. 6, the entire micro syringe infuser (infusion pump 170) can be attached directly to the patient via an adhesive patch 191. The needle set 100 can also be attached directly to the patient via a needle set adhesive dressing 185. As shown in FIG. 5, a wearable belt 190 can house and contain a plurality of micro syringe infusers, such as four separate micro pumps in one exemplary embodiment of the invention. As shown in FIG. 4, the micro syringe infuser 170 may use helical coil springs 173, or small negator mechanisms to apply the force necessary to deliver the infusion fluid from the micro syringe infuser 170 to the needle (via administration set 100).

Variable flow rate infusion systems and methods constructed according to the principles and exemplary embodiments of the invention accurately and reproducibly deliver an infusion fluid to a patient at a desired anatomical location using an infusion pump at a substantially fixed maximum pressure to deliver an infusion fluid to a patient's anatomic site. The infusion fluid is delivered to the anatomic site as fast as permissible by the patient's anatomic site. As the pressure of the anatomic site increases, the flow rate delivered decreases. Patients and clinicians can use the invention to deliver a volume of an infusion liquid at speeds (e.g., flow rate) that do not cause discomfort. The flow rate varies automatically, in response to pressure at the patient's anatomic site, to deliver an infusion fluid at flow rates and pressures that do not cause patient harm. Faster flow rates result from low anatomic site pressures, while slower flow rates result from higher anatomic site pressures. A patient or clinician can set these variables to minimize the amount of pain or discomfort caused by resistance of fluid-filled tissue space and associated pressures during infusion therapies.

Although certain exemplary embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

The claimed invention is:

1. An infusion system for administering an infusion fluid into a patient's anatomic space at a variable flow rate without flow control, the system comprising:
an administration set including:
a flexible tube fluidically connected to a needle connector;
the needle connector including a receiving end fluidically connected to flexible tube, and an administering end opposite the receiving end and fluidically connected with an infusion needle; and
the infusion needle having an inside diameter of about 0.0104 inches to about 0.0135 inches and being fluidically connected to the administering end of the flexible tubing to deliver the infusion fluid to the patient's anatomic space at variable flow rates dependent upon the saturation of the infusion fluid at the patient's injection site.

2. The infusion system of claim 1, wherein the infusion fluid is at least one of the group of an immunoglobulin, a chemotherapeutic fluid, a monoclonal antibody, an antibiotic, an analgesia fluid, and a hydration fluid.

3. The infusion system of claim 1, wherein the flexible tube has an inside diameter of about 0.039 inches to about 0.045 inches.

4. The infusion system of claim 3, wherein the administration set further comprises:
a flexible assembly that conforms to the patient's body to position the needle and reduce the risk of vessel damage.

5. The infusion system of claim 1, further comprising:
an infusion pump configured to deliver infusion fluid at a variable flow rate and at a pressure of about 4 to about 9 psi.

6. The infusion system of claim 5, wherein the infusion pump comprises a compact micro syringe infuser; and
wherein the administration set is connected directly to the compact micro syringe infuser.

7. The infusion system of claim 6, wherein the compact micro syringe infuser includes a fluid reservoir with a volume of about 5-10 ml; and
wherein the administration set is pre-calibrated and includes a length of the tubing measuring about 3 inches to about 7 inches from the micro syringe infuser to the infusion needle.

8. The infusion system of claim 5, wherein the infusion pump is configured to deliver infusion fluid at a variable flow rate and at a pressure of about 4 to about 9 psi.

9. The infusion system of claim 1, wherein the needle connector transitions a size difference from the flexible tubing to the needle to continue laminar flow.

10. The infusion system of claim 1, wherein the infusion system is configured to attach to the patient via an adhesive patch.

11. The infusion system of claim 1, wherein the infusion system includes a garment configured to secure and house the infusion system and worn by the patient.

12. A method for administering an infusion fluid into a patient's anatomic space at a variable flow rate without flow control, the method comprising the steps of:
receiving the infusion fluid from an infusion fluid reservoir at a low pressure into flexible tube;
delivering the infusion fluid from the flexible tube to a receiving end of a needle connector, the needle connector being configured to maintain laminar flow of the infusion fluid;
passing the infusion fluid from the receiving end of the needle connector to an administering end of the needle connector;
delivering the infusion fluid from the administering end of the needle connector to an infusion needle; and
delivering the infusion fluid to the patient's anatomic space at automatically variable flow rates dependent upon the saturation of the infusion fluid at the patient's anatomic space.

13. The infusion method of claim 12, wherein the infusion fluid is at least one of an immunoglobulin, a chemotherapeutic fluid, a monoclonal antibody, an antibiotic, an analgesia fluid, and a hydration fluid.

14. The infusion method of claim 12, further comprising:
delivering the infusion fluid to a plurality of infusion needles.

15. The infusion method of claim 12, further comprising the step of using a flexible assembly that conforms to the patient's body to position the needle and reduce the risk of vessel damage.

16. The infusion method of claim 12, further comprising:
receiving the infusion fluid from an infusion pump with the flexible tube, wherein the infusion pump is configured to deliver the infusion fluid at a variable flow rate and at a pressure of about 4 to about 9 psi.

17. The infusion method of claim 16, wherein the infusion pump comprises a compact micro syringe infuser configured to deliver the infusion fluid at a pressure of about 4 to about 9 psi; and
wherein the flexible tube is connected directly to the compact micro syringe infuser.

18. The infusion method of claim 17, wherein the compact micro syringe infuser includes a fluid reservoir with a volume of about 5-10 ml; and
wherein the combination of the flexible tubing and the infusion needle is pre-calibrated and includes a length of the tubing measuring about 3 inches to about 7 inches from the micro syringe infuser to the infusion needle.

19. The infusion method of claim 12, wherein the flexible tube has an inside diameter of about 0.039 inches to about 0.045 inches and the infusion needle has an inside diameter of about 0.0104 inches to about 0.0135 inches.

20. The infusion method of claim 12, wherein the infusion system is configured to attach to the patient via an adhesive patch.

21. The infusion method of claim 12, wherein the infusion system includes a garment configured to secure and house the infusion system and worn by the patient.
